SEQUENCE LISTING (9 sequences)

SEQ ID NO: 1 - DNA, 702 bp, artificial sequence
  Chemically synthesized
  cDNA (1)..(702): SARS CoV-2 Spike protein receptor binding domain cDNA
gctagcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcggatccggccaccatcaccatcaccac

SEQ ID NO: 2 - protein, 234 aa, artificial sequence
  Chemically synthesized
  MISC_FEATURE (1)..(2)
  DOMAIN (3)..(228): SARS CoV-2 Spike protein receptor binding domain of Wuhan variant
  MISC_FEATURE (229)..(234)
ASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGHHHHHH

SEQ ID NO: 3 - protein, 234 aa, artificial sequence
  Chemically synthesized
  MISC_FEATURE (1)..(2)
  DOMAIN (3)..(228): SARS CoV-2 Spike protein receptor binding domain from Alpha variant
  MISC_FEATURE (229)..(234)
ASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFGSGHHHHHH

SEQ ID NO: 4 - protein, 234 aa, artificial sequence
  Chemically synthesized
  MISC_FEATURE (1)..(2)
  DOMAIN (2)..(228): SARS CoV-2 Spike protein receptor binding protein from Beta variant
  MISC_FEATURE (229)..(234)
ASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGSGHHHHHH

SEQ ID NO: 5 - protein, 234 aa, artificial sequence
  Chemcially synthesized
  MISC_FEATURE (1)..(2)
  DOMAIN (3)..(228): SARS CoV-2 Spike protein receptor binding domain from Gamma variant
  MISC_FEATURE (229)..(234)
ASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFGSGHHHHHH

SEQ ID NO: 6 - protein, 234 aa, Artificial sequence
  Chemically synthesized
  MISC_FEATURE (1)..(2)
  DOMAIN (3)..(228): SARS Co-V Spike protein receptor binding domain from Delta variant
  MISC_FEATURE (229)..(234)
ASRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFGSGHHHHHH

SEQ ID NO: 7 - DNA, 2046 bp, artificial sequence
  chemically synthesized
  cDNA (1)..(2046): SARS CoV2 S1 Domain cDNA
agccagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatccacgtgagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtactatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacaaacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgcagataccacagacgccgtgcgcgacccacagaccctggagatcctggacatcacaccctgctctttcggcggcgtgagcgtgatcacacccggcaccaatacaagcaaccaggtggccgtgctgtatcaggacgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcccagaagagcccggggatccggccaccatcaccatcaccac

SEQ ID NO: 8 - protein, 679 aa, artificial sequence
  Chemically synthesized
  MISC_FEATURE (1)..(2)
  DOMAIN (2)..(673): SARS CoV-2 S1 Domain from Wuhan variant
ASQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSGHHHHHH

SEQ ID NO: 9 - protein, 677 aa, artificial sequence
  Chemically synthesized
  MISC_FEATURE (1)..(2)
  DOMAIN (3)..(671): SARS-CoV-2 S1 Domain from Delta Variant
  MISC_FEATURE (672)..(677)
ASQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSGHHHHHH

(19) United States
(12) Patent Application Publication
(10) Pub. No.: US 2024/0293539 A1
(43) Pub. Date: Sep. 5, 2024

(54) BOVINE COLOSTRUM DERIVED ANTIBODIES AND USES THEREOF

(71) Applicant: ICOSAGEN CELL FACTORY OÜ, Össu, Ülenurme, Tartumaa (EE)

(72) Inventors: Mario PLAAS, Tartumaa (EE); Karin KOGERMANN, Tartumaa (EE); Eva ZUSINAITE, Tartumaa (EE); Toomas TIIRATS, Tartumaa (EE); Birgit AASMÄE, Tartumaa (EE); Ants KAVAK, Tartumaa (EE); Väinö POIKALAINEN, Tartumaa (EE); Lembit LEPASALU, Tartumaa (EE); Sander PIISKOP, Tartumaa (EE); Siimu ROM, Tartumaa (EE); Ruth OLTJER, Tartumaa (EE); Kadri KANGRO, Tartumaa (EE); Eve SANKOVSKI, Tartumaa (EE); Joachim GERHOLD, Tartumaa (EE); Anu PLANKEN, Tartumaa (EE); Raini PERT, Tartumaa (EE); Andres MÄNNIK, Tartumaa (EE); Andres TOVER, Tartumaa (EE); Mihhail KURASIN, Tartumaa (EE); Mart USTAV, Tartumaa (EE); Mart USTAV, Jr., Tartumaa (EE); Kiira GILDEMANN, Tartumaa (EE)

(21) Appl. No.: 18/282,143

(22) PCT Filed: Mar. 14, 2022
(86) PCT No.: PCT/IB2022/052282

§ 371 Date: Sep. 14, 2023

Related U.S. Application Data
(60) Provisional application No. 63/160,833, filed on Mar. 14, 2021.

(51) Int. Cl. A61K 39/395; A61K 9/00; A61K 39/00; A61K 39/42; A61P 37/04

(52) U.S. Cl. CPC A61K 39/39508; A61K 9/0043; A61K 39/42; A61P 37/04; A61K 2039/505; A61K 2039/543; A61K 2039/552

(57) ABSTRACT

A method to produce immunoglobulin preparations against viral infection in humans spreading via respiratory route is provided. The method comprises the steps of immunizing dairy cows during a third trimester of at least a first gestation period with antigen proteins derived from at least one virus strain, collecting hyperimmune bovine colostrum comprising immunoglobulins effective against the antigen protein of various strains of the virus, preparing whey from the colostrum, isolating the immunoglobulin molecules from the whey, and preparing an immunoglobulin preparation for use as an intranasal treatment. One aspect of the invention is to produce SARS-CoV-2 spike protein specific hyperimmune bovine colostrum comprising a high concentration of anti-SARS-CoV-2 antibodies. An intranasal delivery system for diminishing risk of SARS-CoV-2 infections in humans is provided.

### PRIORITY

This application claims priority of U.S. provisional application No. 63/160,833 filed on Mar. 14, 2021 and the contents of which is incorporated herein by reference.

### SEQUENCE LISTING

This application contains a sequence listing provided in computer readable format.

### BACKGROUND

COVID 19-pandemic is a pandemic of coronavirus disease 2019 caused by severe acute respiratory syndrome coronavirus 2 (SARS-CoV-2). SARS-CoV-2 is a novel virus infecting human beings: first human cases of COVID-19 were identified in Wuhan, China, in December 2019.

By now the virus has infected more than 390 million people and at least 5.7 million people have died. The novel coronavirus has had devastating global health, as well as economic consequences and even if there are several vaccine candidates under development the treatment and prevention of the viral infection are still to be developed.

Signs and symptoms of COVID-19 may appear two to fourteen days after exposure. This time after exposure and before having symptoms is called the incubation period. Common signs and symptoms can include fever, cough, tiredness. Other symptoms can include shortness of breath or difficulty breathing, muscle aches, chills, sore throat, runny nose, headache, chest pain. The severity of COVID-19 symptoms can range from very mild to severe. Some people may have only a few symptoms, and some people may have no symptoms at all. Some people may experience worsened symptoms, such as worsened shortness of breath and pneumonia, about a week after symptoms start.

The SARS-CoV-2 virus displays a range of 25-100 homotrimeric Spike (S) proteins on the viral membrane that interact with host cell surface proteins angiotensin-converting enzyme 2 (ACE2), Neuropilin-1 and serine protease TMPRSS2 to facilitate the viral entry to cells (Hoffman et al., 2020, Cantuti-Catelvetri et al., 2020, Daily et al., 2020, Shang et al. 2020). A plethora of antibodies have been reported that bind to various epitopes on the S protein and block the viral entry to cells (Wang et al., 2020, Shi et al., 2020, Noy-Porat et al., 2020, Alsoussi et al., 2020, Liu et al., 2020, Cao et al., 2020, Zost et al., 2020, Trotorici et al., 2020). Most antibodies bind to the receptor binding domain (RBD) of the S protein, that can be found either in upward ACE2 accessible or downward ACE2 inaccessible conformation (Walls et al., 2020) and block the interaction with ACE2. ACE2 is an enzyme attached to the cell membranes of cells located in the lungs, arteries, heart, kidney, and intestines. As a transmembrane protein, ACE2 serves as the main entry point into cells for some coronaviruses, including SARS-CoV-2. More specifically, the binding of the spike S1 protein of SARS-CoV-2 to the enzymatic domain of ACE2 on the surface of cells results in endocytosis and translocation of both the virus and the enzyme into endosomes located within cells.

Neutralizing antibodies can block the entry of a pathogen into the cell and thus prevent the pathogen from infecting the body. As ACE2 is the main entry point for SARS-CoV-2, finding efficient neutralizing antibodies that could block the entry seems a promising approach for developing prophylactic and therapeutic means to fight against the pandemic. Passive immunization with neutralizing anti-SARS-CoV-2 antibody could be especially valuable for certain populations that are suffering the most: the elderly, the immunocompromised, patients in nursing homes and long-term care facilities.

Passing of protective immunity through colostrum in mammals is a naturally evolved process providing protective immunity against exogenous pathogens during the first days and months to the newborn. The use of bovine colostrum as a food supplement has been widely implemented and demonstrated to elicit beneficial effects against intestinal pathogens. In bovine colostrum the main proteins present are immunoglobulins with the main immunoglobulin isotype being IgG, followed by IgA and IgM. The IgG levels in bovine colostrum are around 50-100 mg/ml and this is critical for the calf as cows cannot transfer IgG across the placenta and are dependent of the colostrum in developing the first protective immunity. The maternal serum immunoglobulins decrease rapidly before delivery and are transported into colostrum and milk and thus is an excellent source of immunoglobulins. The development of hyperimmune bovine colostrum can be an excellent source of specific antiviral antibodies. It has been demonstrated in case of influenza where BALB/c mice were pretreated intranasally with IgG obtained from influenza hyperimmune colostrum that protected the mice of development of infection in case of a sublethal dose. The pretreated animals were also protected in case of a lethal infection dose. Similar transfer of hyperimmune colostrum derived protection has also been demonstrated against Clostridium difficile infection.

There is a great need to provide prophylactic treatments and compositions for protection against SARS-CoV-2 infections, and methods for preventing spread of the SARS CoV-2 virus. Not only vaccines are needed, but preventive means that can be modified fast and easily as response to the virus mutations. There is a need to respond fast in case of future pandemics, and even in case of biological war where virus strains spreading via respiratory route may be used. This invention provides method and products to protect humans from such viral infections.

### SUMMARY OF THE INVENTION

Accordingly, it is an object of this invention to provide a method to produce immunoglobulin preparations against viral infection in humans spreading via respiratory route, wherein the method comprises the steps of: immunizing dairy cows during a third trimester of at least a first gestation period with antigen proteins derived from at least one virus strain; collecting hyperimmune bovine colostrum comprising immunoglobulins effective against the antigen protein of various strains of the virus; preparing whey from the colostrum; isolating the immunoglobulin molecules from the whey, and preparing an immunoglobulin preparation for use as an intranasal treatment.

According to certain aspects of the method the immunoglobulin preparation is against SARS-CoV-2 infections and the cows are immunized with at least one SARS-CoV-2 S1 RBD protein, and at least one SARS-CoV-2 spike protein during the third trimester of first gestation period, and optionally during a third trimester of any consequent gestation period, and further optionally reimmunized at least once with at least one SARS-CoV-2 S1 RBD protein between the gestation periods, and wherein the hyperimmune bovine colostrum collected after any parturition comprises immunoglobulins effective against any SARS-CoV-2 strain by blocking entry via ACE2-receptors.

According to certain aspects of the method the cows are immunized during the third trimester of a first gestation period at least once with at least one SARS-CoV-2 S1 RBD protein having at least 90%, preferably at least 95% similarity with any one of the sequences selected from SEQ ID NO:2-6 and with at least one booster comprising SARS-CoV-2 spike protein having at least 90%, preferably at least 95% similarity with SEQ ID: 8 or SEQ ID NO:9; optionally reimmunizing the cow after a first parturition for at least three times with at least one SARS-CoV-2 S1 RBD protein having at least 90% preferably at least 95% similarity with SEQ ID NO:2, and immunizing the cow during a third trimester of a second gestation period at least once with at least one SARS-CoV S1 RBD protein having at least 90%, preferably 95% similarity with any one of sequences selected from SEQ ID NO:2-6, and with at least one booster comprising SARS-CoV-2 spike protein having at least 90%, preferably 95% similarity with SEQ ID: 8 or SEQ ID NO:9; collecting the hyperimmune colostrum after the first and/or the second parturition of the immunized cow.

According to certain aspects of the method the modified hyperimmune colostrum comprises 50-150 mg/ml, preferably 70 to 100 mg/ml of IgG or IgA-type anti-SARS-CoV-2 antibodies.

It is s an object of this invention to provide means for protective immunity in the upper respiratory mucosa against viral infections, especially against SARS-CoV-2 virus infections.

It is an object of this invention to provide an intranasal treatment for humans for use in protection against SARS-CoV-2 by providing and using SARS-CoV-2 Spike protein specific hyperimmune colostrum derived antibodies.

It is an object of this invention to provide a method to produce a SARS-CoV2 specific hyperimmune bovine colostrum, the method comprising the steps of immunizing a dairy cow with at least one SARS-CoV-2 S1 RBD protein variant and at least one SARS-CoV-2 spike protein variant and collecting the modified bovine colostrum after the birth of the calf, wherein the modified bovine colostrum comprises 50 to 150 mg/ml, preferably 70 to 100 mg/ml of anti-SARS-CoV-2 antibodies.

According to certain aspect of the invention a method to produce SARS-CoV-1 specific hyperimmune bovine colostrum is provided, wherein the method comprises the steps of:
immunizing a dairy cow during a third trimester of a first gestation period at least once with at least one SARS-CoV-2 S1 RBD protein having at least 95% similarity with SEQ ID NO:2, and with at least one booster comprising SARS-CoV-2 spike protein having at least 95% similarity with SEQ ID: 4; optionally reimmunizing the cow after a first parturition for at least three times with at least one SARS-CoV-2 S1 RBD protein having at least 95% similarity with SEQ ID NO:2, and immunizing the cow during a third trimester of a second gestation period at least once with at least one SARS-CoV-2 S1 RBD protein having at least 95% similarity with SEQ ID NO:2, and with at least one booster comprising SARS-CoV-2 spike protein having at least 95% similarity with SEQ ID: 4; collecting the hyperimmune colostrum after the first and/or the second parturition of the immunized cow; wherein the hyperimmune colostrum comprises 50-150 mg/ml, preferably 70 to 100 mg/ml of IgG or IgA-type anti-SARS-CoV-2 antibodies.

According to certain aspects in the method of producing the hyperimmune colostrum the cow is immunized during a first gestation period for a first time with the SARS CoV-2 S1 RBD protein 40-70 days, preferably 50-70 days, and most preferably 55-65 days before expected parturition, and a second time 15 to 25 days after the first immunization, and the boost is provided after 10 to 15 days after the second immunization.

According to certain aspects in the method of producing the hyperimmune colostrum the cow is immunized during a second gestation period for 30-50 days, prefrailty 35-45 days, and most preferably 42 days before expected parturition, and the boost is administered after 10-20 days, more preferably 12-15 days, and most preferably 14 days after the last immunization.

According to certain aspects in the method of producing the hyperimmune colostrum the cow is reimmunized between the first and the second gestation period at least three times with SARS CoV-2 S21 RBD protein.

According to certain aspects in the method of producing the hyperimmune colostrum the cow is immunized intramuscularly or via mucosal tissue.

According to certain aspects, the IgG or IgA-type anti-SARS-CoV-2 antibodies of the hyperimmune colostrum is effective against one or more SARS-CoV-2 variants selected from from delta variant Wuhan isolate, UK isolate, South African isolate, Brazilian isolate and Omicron variant.

It is another object of this invention to provide SARS-CoV-2 spike protein specific hyperimmune bovine colostrum collected from a cow immunized with at least one SARS-CoV-2 S1 RBD protein variant and at least one SARS-CoV-2 spike protein variant and the colostrum being collected within 6-10 days after giving birth to a calf and the colostrum comprising 50-150, g/ml, preferably 70 to 100 mg/ml of IgG or IgM type antibodies against at least one SARS-CoV-2 strain.

According to certain aspects the colostrum comprises antibodies effective against at least two different SARS-CoV-2-strains, the strains being selected from Alpha, Beta, Gamma, Delta and Omicron strains.

It is yet another object of this invention to provide a method to make a colostrum immunoglobulin preparation comprising immunoglobulins effective at least against effective against at least two different SARS-CoV-2-strains, the strains being selected from Alpha, Beta, Gamma, Delta and Omicron strains.

According to certain aspect the method to make a colostrum immunoglobulin preparation comprises steps of providing SARS-CoV-2 spike protein hyperimmune bovine colostrum obtained from immunized cows; preparing a whey by removing fat and casein from the colostrum; filtrating the whey; concentrating the whey with tangential flow filtration; purifying the concentrated whey with affinity chromatography and binding immunoglobulin molecules to a G-protein matrix; washing the matrix; eluting the immunoglobulin molecules with glycine at pH 2.7; neutralizing obtained initial immunoglobulin preparation; concentrating the initial preparation; performing buffer exchange on tangential filtration system and sterilizing and filtering the initial preparation to obtain the immunoglobulin preparation.

According to certain aspects, the immunoglobulin preparation made by the method comprises antibodies effective against at least two different SARS-CoV-2-strains, the strains being selected from Alpha, Beta, Gamma, Delta and Omicron strains.

According to certain aspects the immunoglobulin preparation is diluted to comprise 0.01-1 mg/ml, preferably 0.05-0.8 mg/ml, more preferably 0.1-0.5 mg/ml and most preferably 0.15-0.25 mg/ml of anti-SARS-CoV-2 antibodies effective against at least one SARS-CoV-2 strain.

It is yet another object of this invention to provide an intranasal delivery system comprising the immunoglobulin preparation. According to certain aspects the intranasal delivery system is an intranasal spray.

It is a further object of this invention to provide an intranasal delivery system for use in diminishing risk of a human being getting infected by SARS-CoV-2 virus.

According to certain aspects the intranasal delivery system is an intranasal spray for used to spray a dose containing 0.006 to 0.6 mg of anti-SARS-CoV-2 antibodies.

This invention provides a method to produce a large amount of immunoglobulins effective against SARS-CoV-2 by binding to ACE2 receptor. The fast timeline enables fast production of modifications of the intranasal delivery system when new strains of the virus appear.

According to certain aspects the invention provides a method and tools to prepare antiviral protective preparations for fight against any further pandemics.

It is a further object of this invention to provide a method to generate antiviral protective preparations for development of tools against future pandemics or warfare caused by a virus spreading via respiratory route, wherein the method comprises immunizing dairy cows with an antivirus antigen obtained from known virus strains for creating a polyclonal immune response in the cow, collecting colostrum of the cow, isolating immunoglobulin preparation according to claim 8, and providing an intranasal delivery system comprising the immunoglobulin preparation effective against multitude of strains of the virus.

### SHORT DESCRIPTION OF FIGURES

FIG. 1 is a schematic depiction of the workflow for obtaining a hyperimmune colostrum derived antibody containing preparation. The timeline from the first step to the last step can take as little as 2 to 3 months.

FIG. 2 shows antibody titers and includes the depict of antibody neutralization competency. Neutralization titers are indicated by a grey dotted line and grey asterisks. The titer of cow 364 is 1600-fold, cows 1020 and 2279 range around 200-fold. The four remaining cows (2261, 6502, 2261 and 6536) show titers at about 800-fold.

FIG. 3A-E shows immunization and parturition timeline (a) and neutralizing effect of antibodies from serum of the immunized cows 6536 (B, D) and 2279 (C, E) according to the immunization timeline. Graphs B and C show the relative OD450 signal of the ELISA assay performed on serum at 50× dilution. Graphs D and E show the relative OD450 signal of the ELISA assay performed on colostrum at 100× dilution factor.

FIG. 4 the process steps from colostrum to final immunoglobulin product.

FIG. 5 shows the potential of colostrum derived immunoglobulin preparations to block ACE2 binding to Spike protein. Black squares indicate the neutralizing antibody values of cow 6536, black circles those of cow 2279, black triangles of cow 1020 and empty circles depict values for an unimmunized cow (control). Blocking is considered to occur below a relative optical density (OD) of 0.75. The lower the relative OD, the stronger is the neutralizing effect. Colostrum derived immunoglobulin preparations from cow 6536 still show neutralizing competency at a concentration of 32 ug/ml, those of cow 2279 at 78 ug/ml, and those of cow 1020 at a concentration of 125 ug/ml. Colostrum derived immunoglobulin preparations from an unimmunized cow show non-specific neutralization at high concentrations (2500 ug/ml to 10,000 ug/ml).

FIG. 6 illustrates functional characteristics of colostrum derived immunoglobulins from colostrum. Left panel show competition ELISA assay principle. Right panel shows binding of the colostrum immunoglobulin to the different trimeric Speke protein variants in SARS-CoV-2. IC₅₀ values for the variants is also indicated. The colostrum immunoglobulin product was BOSS Ig001, i.e., the product obtained from the colostrum after first parturition (see timeline in FIG. 3A).

FIG. 7 illustrates the functional characteristics of BOSS Ig 001 (product obtained from the colostrum after first parturition) and BOSS Ig 003 (colostrum obtained after second parturition) (see FIG. 3A for the timeline) determined by competitive ELISA. IC₅₀ values are also shown.

FIG. 8 shows the results of a pseudo-virus assay. The assay was conducted on SARS-COV-2 strains B-1-351 LAV (Beta variant), P1 Brazil (Gamma variant), UK202012 (Alpha variant) and Wuhan. Full neutralization is considered at concentrations at which no or practically no luminescence signal is detectable. This is true for all four strains at concentrations around 100 ug/ml, thus indicating equally well neutralization of all the four tested strains. IC50 values are also shown. The test was conducted with BOSS Ig 001 immunoglobulin product.

FIG. 9 shows the results of another set of pseudo-virus assays. Here the tested strains included Alpha, Beta, Gamma, Delta, Kappa and wild type variants. The test was conducted with BOSS Ig 001 immunoglobulin product.

FIG. 10 shows the results of yet another set of pseudo-virus assay. Here the tests included Delta and Omicron variants. The tests were conducted with BOSS Ig 001 and BOSS Ig 003 products.

FIG. 11 illustrates the SARS-CoV-2 neutralization assay and results where SARS-CoV-2 is treated with colostrum derived antibodies in a serial dilution, followed by infection of VERO E6 cells with the virus-antibody mixture. Final antibody concentration where no virus induced cytopathic effects are visually determined, is obtained as a final IC100 concentration. The experiment was performed in separate duplicates and the result of both independent experiments is demonstrated.

FIG. 12 shows the stability and retainment of ACE2 blocking activity of the obtained colostrum derived preparation in different conditions over a 7-day time period. No significant change in activity is detected.

FIG. 13 illustrates viral load changes of a single pre-infection intranasal administration (70 ul) of SARS-COV 2 immunoglobulin product at different protein concentrations. 10⁵ pfu of infectious SARS-COV 2 virus were used to infect the animals.

### DETAILED DESCRIPTION OF THE INVENTION

### Definitions

In this disclosure the terms “immunoglobulin” and “antibody” are used interchangeably.

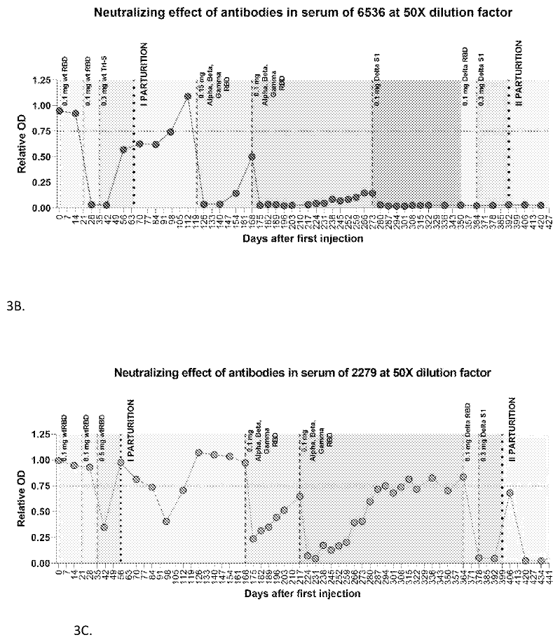

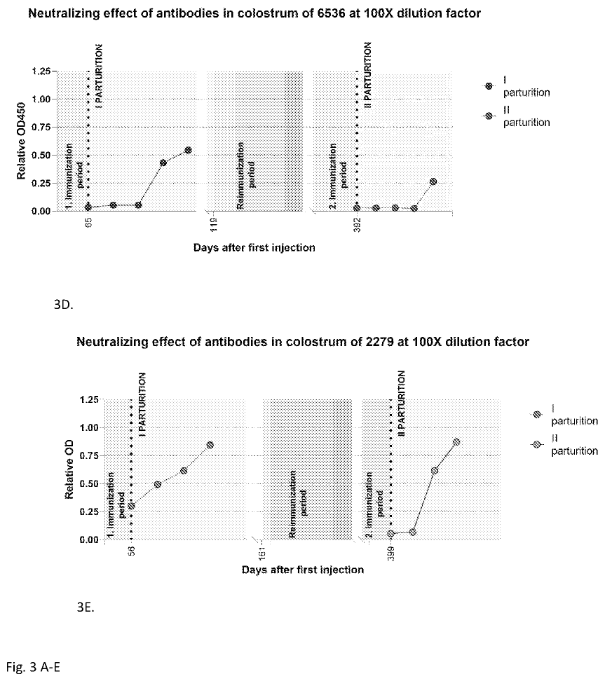

In this disclosure the term “BOSS 001 Ig” means Bovine SARS-CoV-2 Spike Protein Immunoglobulin product obtained from colostrum of immunized cows after the first parturition (see FIG. 3A for timeline of immunizations and parturitions). The term “BOSS 003 Ig” means Bovine SARS-CoV-2 Spike Protein Immunoglobulin product obtained from colostrum of immunized cows after the second parturition (see FIG. 3A for timeline of immunizations and parturitions). In this disclosure, unless otherwise indicated the immunoglobulin product used is BOSS 001 Ig.

In this disclosure the term “colostrum” means the milky fluid obtained from a cow during the first 8-10 milkings after parturition. Milk obtained after that, usually after about 7 days after the parturition is called here “regular milk”.

A method to produce immunoglobulin preparations against viral infection in humans spreading via respiratory route is provided here. The method comprises steps of: immunizing dairy cows during a third trimester of at least a first gestation period with antigen proteins derived from at least one virus strain. The antigen proteins may be selected from any virus spreading via respiratory route, such as but not limited to SARS-Cov, SARS-Cov-2, MERS-CoV. The method comprises collecting hyperimmune bovine colostrum comprising immunoglobulins effective against the antigen protein of various strains of the virus. The immunoglobulins of the hyperimmune bovine colostrum can be effective against other strains than the ones that were used for immunization. In case of immunizing the cows with proteins derived from SARS-Cov-2 Alpha, Beta, Gamma, Delta or Kappa strains, the collected colostrum is also effective against Omicron strain which the cows were never immunized against. From the collected colostrum whey is prepared and the immunoglobulin molecules are isolated from the whey for preparation of an immunoglobulin preparation for use as an intranasal treatment. Preferably the preparation is an intranasal spray.

The method provided here is especially suitable for preparation of an immunoglobulin preparation against SARS-CoV-2 infections. In this case the cows are immunized with at least one SARS-CoV-2 S1 RBD protein, and at least one SARS-CoV-2 spike protein during the third trimester of first gestation period, and optionally during a third trimester of any consequent gestation period, and further optionally reimmunized at least once with at least one SARS-CoV-2 S1 RBD protein between the gestation periods. The hyperimmune bovine colostrum collected after any parturition comprises immunoglobulins effective against any SARS-CoV-2 strain by blocking entry via ACE2-receptors. The hyperimmune colostrum may comprise 50-150 mg/ml, preferably 70 to 100 mg/ml of IgG or IgA-type anti-SARS-CoV-2 antibodies According to one aspect in the method the cows are immunized during the third trimester of a first gestation period at least once with at least one SARS-CoV-2 S1 RBD protein having at least 90%, preferably at least 95% similarity with any one of the sequences selected from SEQ ID NOs: 2-6 and with at least one booster comprising SARS-CoV-2 spike protein having at least 90%, preferably at least 95% similarity with SEQ ID: 8 or SEQ ID NO:9; optionally reimmunizing the cow after a first parturition for at least three times with at least one SARS-CoV-2 S1 RBD protein having at least 90% preferably at least 95% similarity with SEQ ID NO:2, and immunizing the cow during a third trimester of a second gestation period at least once with at least one SARS-CoV S1 RBD protein having at least 90%, preferably 95% similarity with any one of sequences selected from SEQ ID NOs:2-6, and with at least one booster comprising SARS-CoV-2 spike protein having at least 90%, preferably 95% similarity with SEQ ID: 8 or SEQ ID NO:9. The hyperimmune colostrum is collected after the first and/or the second parturition of the immunized cow and it may comprise 50-150 mg/ml, preferably 70 to 100 mg/ml of IgG or IgA-type anti-SARS-CoV-2 antibodies. Surprisingly, the antibodies are effective against any known SARS-CoV-2 virus strain, even against strains that were not used for immunization.

This disclosure provides means for protective immunity in the upper respiratory mucosa against viral infections, especially against SARS-CoV-2 virus infections. An intranasal treatment for humans for use in protection against SARS-CoV-2 is provided by using SARS-CoV-2 Spike protein specific hyperimmune colostrum derived antibodies.

The disclosure provides a preferable immunization schedule for producing the hyperimmune colostrum. The cows are preferably immunized during a first gestation period for a first time with the SARS CoV-2 S1 RBD protein 40-70 days, preferably 50-70 days, and most preferably 55-65 days before expected parturition, and a second time 15 to 25 days after the first immunization, and the boost is provided after 10 to 15 days after the second immunization. The SARS CoV-2 RBD protein may be obtained from any SARS CoV-2 strain, including Alpha, Beta, Gamma, Delta, Kappa or Omicron strain. The SARS-CoV-2 S1 RBD protein preferably has at least 90%, preferably at least 95% similarity with any one of the sequences selected from SEQ ID NOs: 2-6. The immunization schedule induces an immune reaction in the cows that results into the colostrum comprising immunoglobulins that are effective against more than one SARS-CoV strain, even against strains with which the cow was not immunized. Therefore, this method is suitable for diminishing SARS Cov 2 infections in the future caused by new strains not yet known.

The timeline for cow immunization may include immunization during a second gestation or any further gestation period for 30-50 days, prefrailty 35-45 days, and most preferably 42 days before expected parturition, and the boost is administered after 10-20 days, more preferably 12-15 days, and most preferably 14 days after the last immunization. The SARS-CoV-2 S1 RBD protein used for immunization preferably has at least 90%, preferably at least 95% similarity with any one of the sequences selected from SEQ ID NOs: 2-6 The immunization schedule induces an immune reaction in the cows that results into the colostrum comprising immunoglobulins that are effective against more than one SARS-CoV strain, even against strains with which the cow was not immunized.

The immunization timeline may include reimmunization between any two gestation periods for at least once, and more preferably at least three times with SARS CoV-2 S21 RBD protein obtained from any SARS Cov-2 strain. The SARS-CoV-2 S1 RBD protein preferably has at least 90%, preferably at least 95% similarity with any one of the sequences selected from SEQ ID NOs: 2-6.

Any of the immunizations described in this disclosure for producing the hyperimmune colostrum the cow may be intramuscular or via mucosal tissue.

This disclosure provides hyperimmune bovine colostrum that is suitable for isolating immunoglobulins effective against respiratory virus infections, such as SARS-, MERS-CoV and SARS-CoV2 infections. The disclosure provides SARS-CoV-2 spike protein specific hyperimmune bovine colostrum collected from a cow immunized with at least one SARS-CoV-2 S1 RBD protein variant and at least one SARS-CoV-2 spike protein variant. The colostrum is preferably collected from first 8 to 10 milkings after parturition, preferably within 4-10 days after giving birth to a calf and the colostrum can comprise 50-150, g/ml, preferably 70 to 100 mg/ml of IgG or IgM type antibodies against at least one SARS-CoV-2 strain. The colostrum comprises antibodies preferably efficient against SARS-CoV-2 strains different than the strains from which the antigens for immunization were derived. The colostrum preferably comprises antibodies effective against at least two different SARS-CoV-2-strains, the strains being selected from Alpha, Beta, Gamma, Delta, Omicron strains, and any future strains.

This disclosure provides a method to make a colostrum immunoglobulin preparation comprising immunoglobulins effective at least against effective against at least two different SARS-CoV-2-strains, the strains being selected from Alpha, Beta, Gamma, Delta and Omicron strains. The method to make the immunoglobulin preparation preferably comprises steps of providing SARS-CoV-2 spike protein hyperimmune bovine colostrum obtained from immunized cows; preparing a whey by removing fat and casein from the colostrum; filtrating the whey; concentrating the whey with tangential flow filtration; purifying the concentrated whey with affinity chromatography and binding immunoglobulin molecules to a G-protein matrix; washing the matrix; eluting the immunoglobulin molecules with glycine at pH 2.7; neutralizing obtained initial immunoglobulin preparation; concentrating the initial preparation; performing buffer exchange on tangential filtration system and sterilizing and filtering the initial preparation to obtain the immunoglobulin preparation. The preparation comprises antibodies effective against at least two different SARS-CoV-2-strains, the strains being selected from Alpha, Beta, Gamma, Delta, Omicron strains and any future stains. The immunoglobulin preparation may be diluted to comprise 0.01-1 mg/ml, preferably 0.05-0.8 mg/ml, more preferably 0.1-0.5 mg/ml and most preferably 0.15-0.25 mg/ml of anti-SARS-CoV-2 antibodies effective against at least one SARS-CoV-2 strain. The immunoglobulin preparation may be provided as an intranasal delivery system, such as nasal drops, nanogels, intranasal foams or dissolvable packings, dry nasal powder sprays, nasal ointments, and intranasal spray. The intranasal delivery system is for use in diminishing risk of a human being getting infected by SARS-CoV-2 virus. The intranasal delivery system is preferably an intranasal spray for use of doses containing 0.006 to 0.6 mg of anti-SARS-CoV-2 antibodies.

This invention provides a method to produce a large amount of immunoglobulins effective against SARS-CoV-2 by binding to ACE2 receptor. The fast timeline enables fast production of modifications of the intranasal delivery system when new strains of the virus appear. According to certain aspects the invention provides a method and tools to prepare antiviral protective preparations for fight against any further pandemics. The disclosure provides means to generate antiviral protective preparations for development of tools against future pandemics or warfare caused by a virus spreading via respiratory route, wherein the method comprises immunizing dairy cows with an antivirus antigen obtained from known virus strains for creating a polyclonal immune response in the cow, collecting colostrum of the cow, isolating immunoglobulin preparation, and providing an intranasal delivery system comprising the immunoglobulin preparation effective against multitude of strains of the virus. The invention is now described in more details in reference to the figures appended herein. For the development and extraction of hyperimmune bovine colostrum derived SARS-CoV-2 Spike protein specific enriched immunoglobulins, pregnant cows were immunized three times with SARS-CoV-2 Spike protein domains during the third trimester of the first gestation period. In some cases, the cows were re-immunized between first and second parturitions and two immunizations were administered during the third trimester of the second gestation period (see FIG. 3A for immunization timeline).

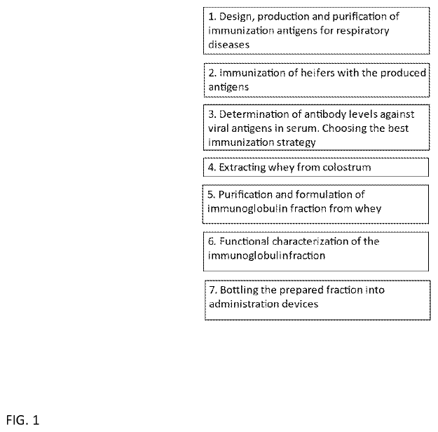

The antigens for immunizations were produced recombinantly in Chinese hamster ovary (CHO-S) cells grown in serum-free chemically defined medium and using QMCF technology (Icosagen Cell Factory OÜ, U.S. Pat. No. 7,790,446, incorporated herein by reference) and purified via C-terminal His-tag using affinity chromatography. After the calves were born the colostrum was collected followed by lipid removal and casein coagulation with chymosin. The resulting whey was further centrifuged, fraction precipitated, heat treated and filtered, resulting in a bovine colostrum derived immunoglobulin containing solution that can be formulated accordingly. FIG. 4 illustrates the process of preparing the final formulation. The obtained solution was then further characterized for the potency to block ACE2 binding in biochemical assays, neutralize SARS-CoV-2 infection in pseudoviral infection assays and neutralize SARS-CoV-2 virus in a cytopathic effect assay (CPE assay). Further the SARS-CoV-2 ACE2 binding blocking antibody solution was formulated and sterile aliquoted in intranasal spray dispenser bottles. The general process overview is depicted in FIG. 1.

### Immunizations of Gestating Cows

Eight cows (Estonian Holstein cattle) in total were immunized 2 times with SARS CoV-2 S1 RBD protein modified from Wuhan isolate (SEQ ID NO:2) in adjuvant solution. We also used SARS CoV-2 S1 RBD protein modified from Alpha variant (SEQ ID NO:3), Beta variant (SEQ ID NO:4), Gamma variant (SEQ ID NO:5), and Delta variant (SEQ ID NO:5). All the used domain proteins were modified similarly so as to have AS-sequence added to N-terminus and HHHHHH-sequence added to C-terminus. After the two immunizations one booster was administered with a SARS-CoV-2 spike protein domain from Wuhan variant (SEQ ID NO: 8) or from Delta variant (SEQ ID NO: 9) in an adjuvant solution. The first immunization occurred 40-70 days, preferably 50-70, more preferably 55-65 days and most preferably 60 days before the expected parturition date for each animal. The second immunization was performed 15 to 25 days, more preferably 20 to 22 days after the first, and the boost was performed 10 to 16 days, more preferably 14 days after the second immunization. The injections were intramuscular, for immunization 0.1 mg of antigen, and for boost 0.5 mg of antigen per injection were used. For adjuvant, Quil-A (0.5 mg/mL, Invivogen)+Imject Alum (10 mg/mL, Thermo) as a mix (3 cows) or Quil-A (0.5 mg/mL, Invivogen) alone were used (5 cows). Instead of intramuscular injections, the immunizations can also be provided as a mucosal spray.

After collection of colostrum after the first parturition, secretion of the SARS CoV-2 neutralizing antibodies into the dairy milk ceased and was at a very low level in the milk. Also, the titer of the neutralizing antibodies in the serum of the animals was at a very low level. Therefore, additional immunization schemes were developed. After the first parturition the cows were re-immunized every xx weeks with SARS CoV-2 S1 RBD protein. During a second gestation period the cows were immunized 30-50, more preferably 35-45, and most preferably 42 days before the expected parturition date. A boost with SARS-CoV-2 spike protein domain was given 10-20 days, more preferably 12-15 days and most preferably 14 days after the last immunization.

FIG. 3A shows the immunization timeline.

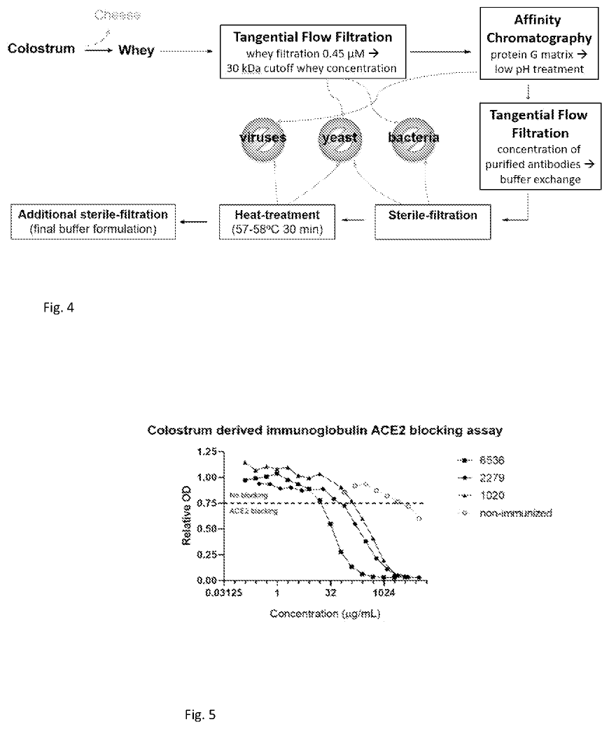

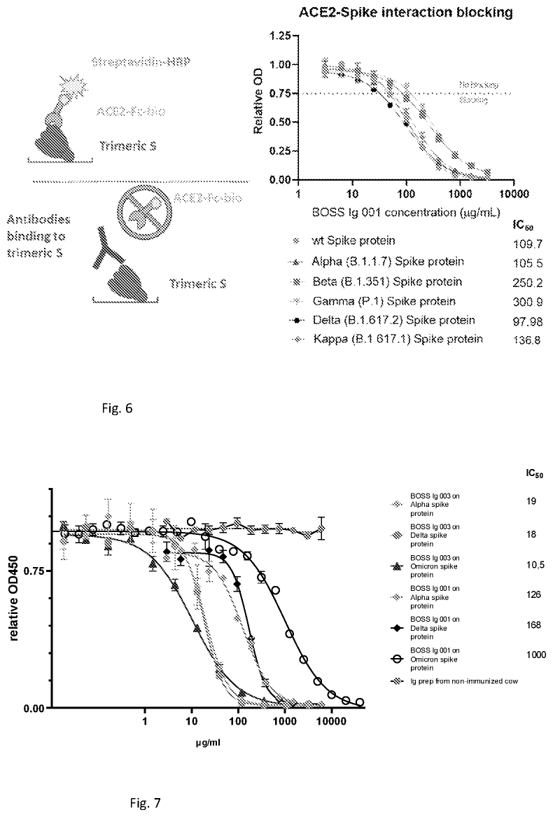

RBD, S1 and trimeric S proteins were produced by generating gene sequences and respective expression vectors (QMCF-vectors and -technology; c.f. Silla et al. 2005 J. Virol. Vol. 79, No. 24; U.S. Pat. No. 7,790,446) for protein production in CHO cells. The produced proteins of each viral isolate were purified to homogeneity and used for further immunization of lactating cows. The cows received immunizations with 2 or 3 doses during the lactation period. All cows received a mix of equal amounts of Alpha, Beta and Gamma strain RBD proteins as antigens in an adjuvant solution. Some cows also received Delta strain S1 protein as an antigen in an adjuvant solution. In the last trimester of second gestation period the cows were immunized once with SARS-CoV-2 Delta strain RBD protein in adjuvant solution followed by one boost with Delta strain S1 protein in adjuvant solution. The immune response was monitored by collecting serum every 2-3 weeks and analyzing it with a competition ELISA assay to detect neutralizing antibodies, antibodies capable of blocking the interaction between trimeric S protein and its receptor molecule ACE-2. Principle of the competition ELISA assay is shown in FIG. 6, left panel.

Immunized Animal Serum Response Against SARS-CoV-2 Spike Protein after the First Immunization

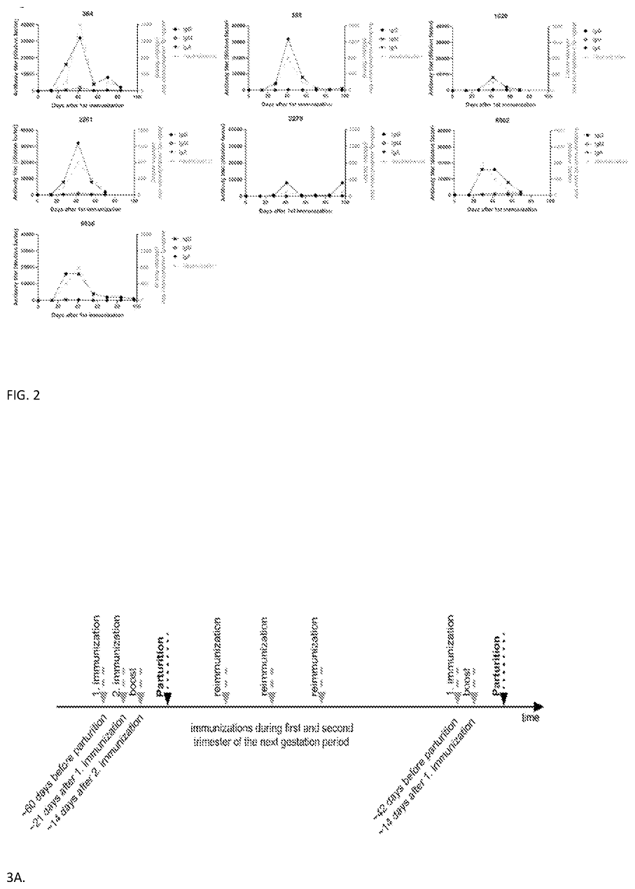

In order to monitor the occurrence of a SARS-CoV-2 Spike protein specific immune response after the first immunization, blood sera from the test animals were obtained before and during the immunizations until calving. A strong onset of Spike specific immune response was observed starting from day 25 post immunization and peaking around day 40 as demonstrated in FIG. 2. Results are shown for 7 cows. The majority of the detected serum immune response was IgG immunoglobulins with a low detection of IgM serum antibodies for some test animals at day 40. For determining the ability of the obtained serum response to potentially neutralize the SARS-CoV-2, a biochemical ACE2 blocking assay was performed. S-protein (e.g. SEQ ID NO: 4 was coated at 2.5 pg/mL on a MaxiSorp high-capacity binding ELISA plate (Thermo Fisher Scientific, US). S-protein was incubated with serially diluted serum obtained from the test animals at different time points, followed by washes and incubation with biotinylated ACE2-Fc and detection by Strepavidin-HRP. A minimal dilution factor where ACE2 blocking was evident is indicated on the right Y-axis in FIG. 2.

### Immunized Animal Serum Response Against SARS-CoV-2 Spike Protein During Full Immunization Schedule

FIG. 3A-E shows immunization and parturition timeline (A) and neutralizing effect of antibodies from serum of two immunized cows (B for 6536 and C for 2279) according to the full immunization schedule. Graphs B and C shown the relative OD450 signal of the ELISA assay performed on serum at 50× dilution, lowest signals meaning antibodies with high capability of binding trimeric S protein of SARS-CoV-2, thus blocking its interaction the ACE-receptor. Graphs D (cow 6536) and E (cow 2279) show the relative OD450 signal of the ELISA assay performed on colostrum at 100× dilution factor.

The results clearly show there is a clear effect of immunization and re-immunization on the neutralizing effect of antibodies from the serum of the immunized cows. After each dose of antigens, measurable antibody ability to block ACE2-trimeric S-protein interaction clearly increased. The positive effect of re-immunization can also be seen in the colostrum (FIGS. 3D and E). Neutralizing antibodies were detected even in the 5^{th} milking of cow 6536 and in first 3 milkings in cow 2279. Reimmunization of the cows resulted in higher neutralizing effect.

It was also found that continuing reimmunization of every 5 to 7 weeks lead to detection of neutralizing antibodies in concentrated whey of regular milk (results not shown).

Accordingly, this disclosure provides a method to obtain from colostrum of immunized dairy cows efficient neutralizing antibodies capable of blocking ACE2-trimeric S protein. A method to produce a hyperimmune (modified) bovine colostrum is provided, where the method comprises the steps of immunizing a dairy cow with at least one SARS-CoV-2 S1 RBD protein variant and at least one SARS-CoV-2 spike protein variant and collecting the modified bovine colostrum after the birth of the calf, and even up to 3-5 milkings of the cow after the parturition. In a preferred embodiment the obtained hyperimmune colostrum comprises 70 to 100 mg/ml of anti-SARS-CoV-2 antibodies as IgG immunoglobulins.

According to the method provided in this disclosure the cows are immunized for a first time with SARS CoV-2 S1 RBD protein 40-70, preferably 50-70, and most preferably 55-65 days before expected parturition, and a second time 15 to 25 days after the first immunization, and the SARS-Cov-2 spike protein boost is provided after 10 to 15 days after the second immunization.

The modified (hyperimmune) colostrum that was proved to contain the immunoglobulins was processed further as described below.

Preparation of Immunoglobulin-Enriched Solution from Bovine Colostrum

Colostrum was collected for 4 to 6 days after the birth of the calves. The first colostrum after the birth contained the highest antibody concentration amounting to 70-100 mg/ml, and the amount of the milk was about 20 liters. Fat and casein were removed from the milk by coagulation with chymosin, and the resulting whey was further processed as described below, and illustrated in FIG. 4.

Whey that was separated from the cows milk is first filtrated with 0.45 μM filters to remove yeasts, bigger bacteria and other particles. Next the tangential flow filtration method with 30 kDa cutoff cassettes is used to concentrate whey up to 10 times. Concentrated whey is then purified with affinity chromatography, using G-protein matrix. During chromatography purification the antibodies are bound to the matrix, which is then washed with 1×PBS solution to eliminate any whey residues. Second wash is performed with 850 mM NaCl in 1×PBS solution, to weaken non-specific interaction (protein-protein, protein-surface) to eliminate contaminants. The immunoglobulin fraction is eluted with 0.1M Glycine, pH 2.7. To eliminate possibly present viruses from the preparation, low pH treatment is performed by leaving the purified material in glycine buffer for 30 minutes. The IgG preparation is then neutralized with 1.5M Tris buffer, pH 7.5. The purified antibody solution is then concentrated, and buffer exchange is performed on tangential flow filtration system with 50 kDa cutoff filters. Next the final product is sterile-filtered and heat-treated at 58° C. for 30 minutes. Sterile filtration was then conducted through 0.22 μm filters to obtain the final formulation. The filtration formulation is called here BOSS Ig (Bovine SARS-Cov-2 Spike Protein Immunoglobulins).

Alternatively, defatted casein-depleted whey was frozen at −20° C. and held until purification step. To remove casein remains the whey was melted at +4° C., then pH was adjusted to 4.2-4.5 using 1 M HCl and the whey was left at room temperature for 1 hour with continuous mixing using magnet stirrer. Thereafter pH was adjusted to 3.3 with 1 M HCl and the whey was left for 1 hour at room temperature to inactivate possible viral contaminants. The pH-treated colostral whey was neutralized to pH value 6.7-7.0 using 1.5 M Tris-HCl pH 8.8 and filtrated through 5 μm and 0.22 μm filters. Filtrated solution was mixed with ammonium sulphate to final concentration 2 M to initiate precipitation of colostral proteins and the precipitation procedure was carried out at +4° C. overnight. Next day protein precipitate was separated from supernatant by centrifugation at 7000×g for 15 min at 4° C. The precipitated colostral proteins were resuspended in 1×DPBS (Dulbecco's phosphate-buffered saline) solution and concentrated to 100 mg/mL. Concentration was measured by reading absorbance at 280 nm and using IgG absorbance for 0.1% solution (A₂₈₀^{0.1%})=1.37. Concentrated immunoglobulin-enriched solution was dialyzed against 1×DPBS by means of tangential flow filtration (TFF). TFF was carried out on Äkta Flux 6 using Sartocon cassettes with 50 kDa cutoff number. Dialyzed colostral proteins were 0.22 μm filtrated followed by pasteurization. Pasteurization step was done by heating the solution at 63° C. for 30 minutes followed by sterile filtration through 0.22 μm filters. The resulted filtration product was then tested further. Microbiological quality of the BOSS Ig was tested in accordance with European Pharamacopoeia 10.3 methods 2.6.1. Sterility testing and 2.6.12. Microbial examination testing. The tests showed that no growth of microorganisms occurred, and the BOSS Ig preparations passed the testing and complied with the test for sterility (results not shown).

Now the BOSS Ig product was further tested for ACE2 blocking activity as described below:

### Characterization of the ACE2 Blocking Activity of the Colostrum Derived Immunoglobulin Preparation Using Biochemical Assay to Measure the Inhibition of the Viral Spike Protein and Human Cellular Receptor ACE2 Interaction.

Spike protein of the SARS-CoV-2 virus contains the binding region of the virus receptor, which mediates the adsorption and entry of the virus into cells via human receptor ACE2. Thus, the inhibition of the viral entry can be achieved by inhibition of the interaction between S protein and ACE2.

FIG. 6 (left panel) depicts the principle of the competition ELISA assay used to determine the ACE2-trimeric S protein interaction by the added immunoglobulin preparation. FIG. 6 (right panel) shows a graph of the ACE2-trimeric spike protein interaction blockage capacity of the initially obtained immunoglobulin preparation (BOSS Ig 001) Briefly, ELISA plate wells were coated with spike protein of the SARS-CoV-2 of respective variant of concern (VOC). Then the sample of interest (here colostrum immunoglobulin preparation) was added and pre-incubated to allow the antibodies to bind. Next, the enzyme complex containing biotin-labelled extracellular domain of ACE2 (human receptor for cell entry of SARS-CoV-2 virus) and HRP-labelled streptavidin was added without any washes in between and incubated for 30 minutes). After washing out unbound ACE2, the spike-ACE2 interaction was detected using TMB substrate. In interpretation of competition ELISA results, the higher signal indicates more interaction between the viral spike protein and human ACE2 receptor, and lower signal thus indicates blocking of the ACE-trimeric S-interaction and therefore the likely presence of neutralizing antibodies. The inhibition of this interaction by colostrum immunoglobulin preparation is expressed in relative optical density (OD) units and calculated as ratio of OD value in the sample of interest divided by OD value in the sample without the inhibitor. Thus, lower relative OD value indicates more inhibition of the interaction between the viral spike protein and human ACE2 receptor. For calibration of the system well characterized monoclonal antibodies against SARS-CoV-2 and human convalescent plasma from individuals recovered from COVID-19 were used, and the relative OD values less than 0.75 were deemed to be indicative for the inhibition.

In FIG. 5, the results are shown when the inhibition properties of the 3 different immunoglobulin preparations (cows 6536, 2279, 1020) from the colostrum of cows immunized with the SARS-CoV-2 spike protein from Wuhan variant were analyzed in the assay described above. FIG. 6 right side panel shows similar results of immunoglobulin preparation tested on Wuhan, Beta, Gamma, Delta and Kappa variants. Importantly, here the cows were immunized with CoV-2 spike protein of Wuhan variant only, but the neutralizing capacity of the obtained immunoglobulin preparation was detected on all the tested variants although in different efficiencies. The 150 values for the different variants as shown in FIG. 6 are: approximately 100 ug/ml for Wuhan, Alpha and Delta variants, 130 ug/ml for Kappa variant and 250-300 ug/ml for Beta and Gamma variants. Clearly, the preparations inhibit the interaction between the spike-protein and ACE2 in concentration dependent manner and thus have effect to block the viral entry to the human cells. In contrast, there was no such inhibiting activity observed when using the control preparation from the colostrum of non-immunized cow (shown in FIG. 5).

Moreover, it is clear that the immunoglobulin preparation is efficient against variants other than the one used for the immunization. To further evaluate this aspect of the invention, the experiments were expanded to recent arrival of Omicron variant:

We tested BOSS Ig 001 immunoglobulin product and BOSS Ig 003 on Alpha, Delta and Omicron spike proteins. Results are shown in FIG. 7. While IC50 values of BOSS Ig 001 for Aplha and Delta were 126 and 168 ug/ml, respectively, IC 50 value for Omicron variant was as high as 690 ug/ml. However, with BOSS Ig 003 the IC 50 values of Alpha, Delta and Omicron were 19 ug/l; 18.5 ug/ml and 10.5 ug/ml, respectively. This shows that the continuing immunization of the animals clearly increased polyclonal potency of the preparation.

Accordingly, this disclosure provides a method to obtain provide hyperimmune colostrum from immunized dairy cow. According to one aspect the method comprise the steps of immunizing a dairy cow with at least one SARS-CoV-2 S1 RBD protein variant and at least one SARS-CoV-2, collecting the hyperimmune colostrum after first parturition and optionally continuing immunizations every 5 to 7 weeks after the first parturition, and providing further immunizations during the third trimester of a second gestation period, and further collecting the colostrum after the second parturition, and preparing immunoglobulin products from the colostrum after the first and/or the second parturition. In a preferred embodiment the product has a high neutralizing effect of ACE2 trimeric protein of more than one variants, and the IC 50 value for the variants ranges between 200 to 20 ug/ml. According to one aspect of the invention, the colostrum comprises immunoglobulins efficient in blocking ACE2-trimeric S proteins of multitude of SARS CoV-2 variants. According to certain aspects of the invention, the colostrum comprises immunoglobulins capable of blocking ACE2-trimereic S proteins of SARS CoV-2 variants other than the variants that were used in immunization of the cows.

Characterization of the Antiviral Activity of the Colostrum Preparations in a Pseudoviral Assay that Mimics the Viral Entry During Infection of the SARS-CoV-2

To further characterize the virus neutralizing potency of the colostrum immunoglobulin preparations, the pseudoviral neutralization assay was performed. The pseudovirus refers to a retrovirus that carries or is pseudotyped with the envelope glycoprotein of another virus to form a virus with an exogenous viral envelope while the genome retains the characteristics of the retrovirus itself. Here, an HIV-based pseudovirus containing a luciferase marker gene incorporated into the proviral DNA was used to characterize antiviral activity of the colostrum preparations.

The ability of the HIV-based pseudovirus to infect the ACE2 containing HEK 293-cells in a single round was shown to be strictly dependent on the presence of the pseudotyped SARS-CoV-2 spike protein on the surface of the pseudoviral particles. The quantizable luciferase marker gene becomes integrated into the target cell genome after infection. Thus, the effectiveness of the spike protein dependent infection can be measured by luciferase activity becoming detectable in the target cells after successful infection of the pseudovirus and the inhibition of viral entry into cells is correlated to the decreased levels of luciferase signals. It is generally considered that such assay is highly relevant to evaluate the antiviral properties of the different compounds, like immunoglobulins.

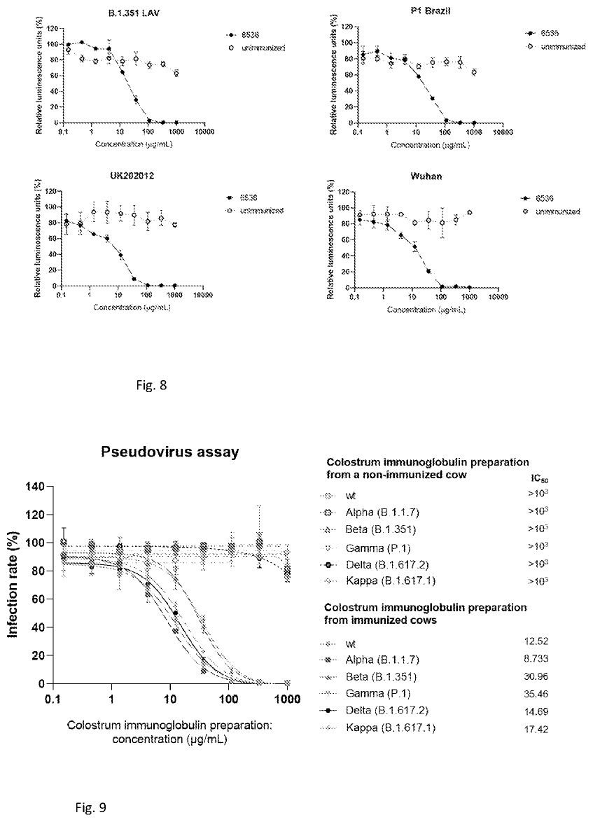

In FIG. 8, the results of the above described pseudovirus assays are shown using immunoglobulin preparation derived from the colostrum of the SARS-CoV-2 spike protein immunized cow (6536) or from an unimmunized cow. Different pseudoviruses were used that were pseudotyped with the spike protein of different SARS-CoV-2 virus variants widely and rapidly spreading in 2020/2021 season (South-Africa lineage B.1.351, Brazil lineage P1, United Kingdom variant UK202012/01 and original Wuhan virus). Clearly, the preparation derived from the immunized cow efficiently inhibited the entry of all pseudoviruses tested here and complete inhibition was observed at protein concentration around 100 pg/ml. In contrast, the colostrum immunoglobulins from the unimmunized cow have no effect. FIG. 9 shows similar experiments with spike protein from Alpha, Beta, Gamma, Delta and Kappa variants. IC₅₀ values in this test for Alpha variant was 8.733, for Beta 30.96, for Gamma 35.36, for Delta 14,69 and for Kappa 17.42. Colostrum immunoglobulin from non-immunized cow showed no inhibition effect.

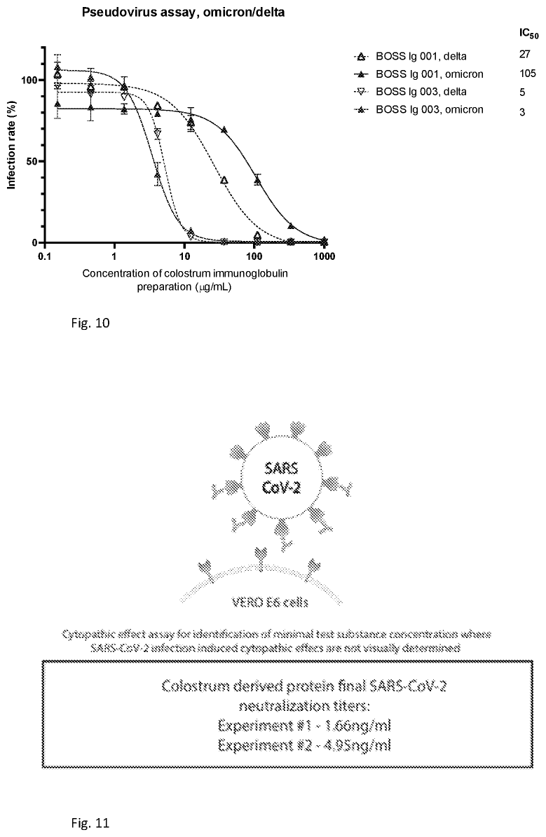

The experiment was again extended to new Omicron variant. FIG. 10 shows an assay for BOSS 001 and BOSS 003 on Delta and Omicron variants. In preparation of BOS 001 the cows were reimmunized with Alpha, Beta, Gamma and Delta variants, but not with Omicron. As can be seen the immunoglobulin preparation BOS 003 obtained from colostrum after second parturition of reimmunized cows is significantly more effective on both Delta and Omicron variants with IC₅₀ values of 5 and 3 ug/ml, respectively. Thus, the BOSS 003 shows about 10 times higher efficiency on Omicron variant as compared to BOSS 001. The fact that the inhibition was clearly seen on Omicron variants even if the cows were never immunized with Omicron proteins shows that the continuing reimmunization provides much more matured immune response and consequently a polyclonal response.

Accordingly, this disclosure provides a method to produce immunoglobulin product from hyperimmune colostrum of immunized cows effective against multitude of viral variants. In one aspect of the invention this disclosure provides a method to produce immunoglobulin product from colostrum of reimmunized cows for inducing immune response against common epitopes in SARS CoV 2-viral isolates and the immune response can extend to variants that have not been used for immunizing the cows and therefore the invention provides a method to provide immunoglobulin product effective against currently existing as well as future SARS CoV-2 variants. The disclosure thus provides a method to produce antibodies that are effective against the known SARS Cov-2 isolates but also to still unknown variants. In its broadest scope this invention provides a method to produce immunoglobulin products to avoid further pandemics or even for protection against biological weapons.

### Identification of Live Virus Neutralization Potency Using VERO E6 Assay

To further characterize the virus neutralizing potency of the colostrum immunoglobulin preparations not only for spike-ACE2 interaction dependent initiation of the cell entry but more generally in aspect of the authentic virus replication in the target cell population, well known Vero E6 assay was used for cell based live virus neutralization assay. Vero E6 cell line is derived from the kidney of African green monkey and the cells have high level of ACE2 expression and are highly susceptible to wt SARS-CoV-2 infection. Thus, in those assays, even minimal infection led to replication and spread of the virus in the cell culture and cytopathic effect was easily detectable by microscopy. In contrast, lack of the cytopathic effect in the presence of an antiviral agent (e.g. viral neutralizing immunoglobulin) indicates complete neutralization and protection in the assay.

The virus neutralizing properties of the immunoglobulins derived from the colostrum of the cows (6536, 2279) immunized with the spike protein of SARS-CoV-2 were characterized in the Vero E6 assay. Briefly, the SARS-CoV-2 (Estonian isolate 3542) was diluted in DMEM/0.2% BSA/Pen-Strep and 10² pfu of the virus was added of serially diluted colostrum immunoglobulin preparations. Mixtures were incubated for 1 h at 37° C. Then suspension of Vero E6 cells was added to the wells of a 96 well tissue culture plate in triplicates (4×10e4 cells/well) at a final volume 100 ul/well. The virus treated cells were incubated at 37° C., 5% CO2 for 5 days. Then the results of neutralization assay were evaluated microscopically by detecting appearance of cytopathic effect. The first dilution (measured by total protein concentration of the preparation), where cytopathic effect was absent was determined as the completely neutralizing concentration. In different experiments, the complete virus neutralization was observed in concentrations as low as 0.8-2.2 ng/ml. FIG. 11 illustrates the results of two SARS-CoV-2 virus neutralization assays, that determines minimal test sample concentrations where no SARS-CoV-2 induced cytopathic effects of Vero E6 cells are visible, where complete virus neutralization was observed at sample concentrations of 1.66 ng/ml and 4.95 ng/ml, respectively.

### Animal Experiments

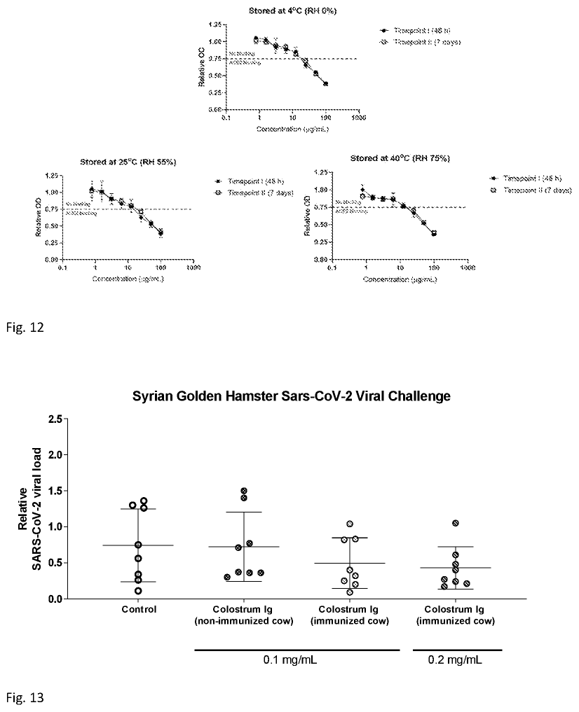

In order to evaluation the in-vivo efficacy of the developed immunoglobulin containing formulation a prophylactic study as performed by administering intranasally SARS VoV-2 formulation BOSS Ig 001 at different concentration to Syrian Golden Hamsters, followed by an infection of 105 pfu of SARS-COv-2 virus. The infected animals were monitored for 4 days after which they were sacrificed and lung viral titer was determines. As is seen in FIG. 13, the non-hyperimmune antibody preparation (colostrum Ig non immunized cows) did not have an effect on reduction of the viral titer. Both tested concentration of BOSS Ig 001 (0.1 mg/ml and 0.02 mg/ml) had an efficacy in reducing the high viral load even after a single intranasal administering dose.

Accordingly, an aspect of this invention is to provide an immunoglobulin preparation obtained from hyperimmune colostrum of immunized cows for use as a nasal administration system to provide a protective effect in reducing SARS CoV-2 infections.

### Evaluation of Functional Stability of the Antibody Preparation in Time in Various Conditions

In order to evaluate the stability of the obtained colostrum immunoglobulin preparations the immunoglobulin containing solution was incubated at 3 different temperatures 4° C., 25° C. and 40° C., over a 7 day period and ACE2 blocking activity was measured 48 h and 7 days post incubation. As seen from FIG. 12 no significant change of ACE2 blocking activity was determined over time, indicating high functional stability of the obtained antibody preparations. In further experiments it was found that the BOSS Ig-products remain stable for at least 11 months when stored at +4° C. or −20° C. (results not shown). Further experiments have showed that the antibodies in final intranasal spray remain effective for at least 9 months (results not shown). Accordingly, this disclosure provides colostrum immunoglobulin preparations that remain stable for at least 11 months at +4° C.

### Use of the Colostrum Derived Antibodies in Health Care

Certain aspects of this invention include colostrum immunoglobulin preparations for use to lower a risk of SARS-Cov-2 infection in a human. According to a preferred embodiment the colostrum immunoglobulin preparations are for use in intranasal delivery systems. Preferable intranasal delivery system of the colostrum immunoglobulins or their combinations is solution sprays. According to certain aspects the colostrum immunoglobulin products may also be selected from nasal drops, nanogels, intranasal foams or dissolvable packings, dry nasal powder sprays, and nasal ointments.

In making immunoglobulin preparations for intranasal delivery systems the colostrum immunoglobulin product obtained from colostrum and purified as described above is used as a starting material. In the final product pH is adjusted and the immunoglobulin concentration is adjusted as desired for example with DPBS pH 7.4. The solution is pasteurized, sterilized and packed for use. The intranasal delivery system may comprise for example 0.01-1 mg/ml, preferably 0.05-0.8 mg/ml, more preferably 0.1-0.5 mg/ml and most preferably, 0.015-0.25 mg/ml of anti-SARS CoV-2 antibodies.

In order to determine how long the colostrum intranasal delivery system persists on the nasal mucosa, healthy individuals were invited to participate a clinical trial. Eight individuals were administered an intranasal spray formulation containing BOSS Ig 001 with a final concentration of 0.2 mg/ml. Each nostril was administered with 2 doses about 100 ul each, thus a total of 40 ug colostrum immunoglobulins. Thereafter, filter paper pieces with a volume capacity of 15 ul were placed onto the nasal mucosa after 5 minutes, 1 h and 4 h after the administration. The paper pieces were kept for 10 minutes to absorb bovine IgG from the mucosa. As a control, samples were taken prior to the administration of the spay. Samples were analyzed using a bovine-IgG specific kit (Abcam, Cow Ig ELISA kit, cat No ab 190517). It was observed that bovine IgG could be detected in all individuals after 1 h with IgG at an average concentration of 5.65 ug/l was detectable. After 4 hours 2.36 ug/l was detectable. Thus, according to one aspect of the invention a colostrum immunoglobulin product, preferably an intranasal spray is provided, wherein the product comprises 0.01-1 mg/ml, preferably 0.05-0.8 mg/ml, more preferably 0.1-0.5 mg/ml and most preferably 0.15-0.25 mg/ml of anti-SARS-CoV-2 antibodies. Using the intranasal spray prophylactically could include spraying a dose containing 0.006 to 0.6 mg of immunoglobulins before attending a situation with increased risk to get infected. Use of the preparation provides a protection for 8 to 15 hours.

In this disclosure a method to produce colostrum comprising high concentrations of anti-SARS-CoV-2 antibodies is provided especially for use in intranasal spray. The immunoglobulin spray product may contain for example 0.2 mg/ml of anti-SARS-CoV-2 antibodies isolated from colostrum of dairy cows immunized once or for multiple times with RBD, S1 and/or trimeric S protein of various viral isolates, including alpha, beta, gamma, delta and omicron variants. The product may contain antibodies effective against multiple virus strains. In the experimental part immunization of the cows with antigens from few virus strains is described. However, a skilled artisan understands that the invention includes using any new strains of the virus to isolate or chemically synthesize the sequences for immunizing the cows. This flexibility provides a fast method to respond to the fast evolution of the virus. Moreover, the method and the product described here provides tools for inhibiting new variants not yet known and thus a tool for fighting new pandemics.

What is claimed is:

1. A method to produce immunoglobulin preparation against viral infection in humans spreading via respiratory route, wherein the method comprises the steps of:
immunizing dairy cows during a third trimester of at least a first gestation period with antigen proteins derived from at least one virus strain;
collecting hyperimmune bovine colostrum comprising immunoglobulins effective against the antigen protein of various strains of the virus;
preparing whey from the colostrum;
isolating the immunoglobulin molecules from the whey; and
preparing an immunoglobulin preparation for use as an intranasal treatment.

2. The method of claim 1, wherein immunoglobulin preparation is against SARS-CoV-2 infections and the cows are immunized with at least one SARS-CoV-2 S1 RBD protein, and at least one SARS-CoV-2 spike protein during the third trimester of first gestation period, and optionally during a third trimester of any consequent gestation period, and further optionally reimmunized at least once with at least one SARS-CoV-2 S1 RBD protein between the gestation periods, and wherein the hyperimmune bovine colostrum collected after any parturition comprises immunoglobulins effective against any SARS-CoV-2 strain by blocking entry via ACE2-receptors.

3. The method of claim 1, wherein the cows are immunized during the third trimester of a first gestation period at least once with at least one SARS-CoV-2 S1 RBD protein having at least 90%, preferably at least 95% similarity with any one of the sequences selected from SEQ ID NO:2-6 and with at least one booster comprising SARS-CoV-2 spike protein having at least 90%, preferably at least 95% similarity with SEQ ID: 8 or SEQ ID NO:9;
optionally reimmunizing the cow after a first parturition for at least three times with at least one SARS-CoV-2 S1 RBD protein having at least 90% preferably at least 95% similarity with SEQ ID NO:2, and immunizing the cow during a third trimester of a second gestation period at least once with at least one SARS-CoV S1 RBD protein having at least 90%, preferably 95% similarity with any one of sequences selected from SEQ ID NO:2-6, and with at least one booster comprising SARS-CoV-2 spike protein having at least 90%, preferably 95% similarity with SEQ ID: 8 or SEQ ID NO:9;
collecting the hyperimmune colostrum after the first and/or the second parturition of the immunized cow;
wherein the modified hyperimmune colostrum comprises 50-150 mg/ml, preferably 70 to 100 mg/ml of IgG or IgA-type anti-SARS-CoV-2 antibodies.

4. The method of claim 3, wherein during a first gestation period the cow is immunized for a first time with the SARS CoV-2 S1 RBD protein 40-70 days, preferably 50-70 days, and most preferably 55-65 days before expected parturition, and a second time 15 to 25 days after the first immunization, and the boost is provided after 10 to 15 days after the second immunization.

5. The method of claim 3, wherein the cow is immunized during a second gestation period for 30-50 days, preferably 35-45 days, and most preferably 42 days before expected parturition, and the boost is administered after 10-20 days, more preferably 12-15 days, and most preferably 14 days after the last immunization.

6. The method of claim 2, wherein the cow is reimmunized between the first and the second gestation period at least three times with SARS CoV-2 S21 RBD protein.

7. The method of claim 1, wherein the cow is immunized intramuscularly or via mucosal tissue.

8. A SARS-CoV-2 Spike protein specific hyperimmune bovine colostrum produced according to claim 1 and collected after the first or the second parturition and comprising 70 to 100 mg/ml of IgG or IgA-type anti-SARS-CoV-2 antibodies being effective against at least one SARS-CoV-2 strain.

9. The hyperimmune bovine colostrum of claim 8, wherein the colostrum comprises antibodies effective against at least two different SARS-CoV-strains, the strains being selected from Alpha, Beta, Gamma, Delta and Omicron strains.

10. A method to make a colostrum immunoglobulin preparation, the method comprising the steps of:
providing hyperimmune bovine colostrum according to claim 8;
preparing a whey by removing fat and casein from the colostrum;
filtrating the whey;
concentrating the whey with tangential flow filtration;
purifying the concentrated whey with affinity chromatography and binding immunoglobulin molecules to a G-protein matrix;
washing the matrix;
eluting the immunoglobulin molecules with glycine at pH 2.7;
neutralizing obtained initial immunoglobulin preparation;
concentrating the initial preparation;
performing buffer exchange on tangential filtration system and
sterilizing and filtering the initial preparation to obtain the immunoglobulin preparation.

11. A colostrum immunoglobulin preparation made by the method according to claim 10.

12. The colostrum immunoglobulin preparation of claim 11, wherein the preparation is diluted to comprise 0.01-1 mg/ml, preferably 0.05-0.8 mg/ml, more preferably 0.1-0.5 mg/ml and most preferably 0.15-0.25 mg/ml of anti-SARS-CoV-2 antibodies effective against at least one SARS-CoV-2 strain.

13. The colostrum immunoglobulin preparation of claim 11 effective at least against two SARS-CoV-2 strains.

14. The colostrum immunoglobulin preparation of claim 13, wherein the strains are selected from Alpha, Beta, Gamma, Delta, Omicron and any novel SARS CoV-2 strains.

15. A mucosal intranasal delivery system comprising the colostrum immunoglobulin preparation of claim 11.

16. The mucosal intranasal delivery system of claim 15, wherein the system is an intranasal spray.

17. (canceled)

18. A method to diminish a risk of a human being to get infected by SARS-CoV-2 virus, the method comprising administering the spray of claim 16.

19. A method to generate antiviral protective preparations for development of tools against future pandemics or warfare caused by a virus spreading via respiratory route, wherein the method comprises
preparing an immunoglobulin preparation for an intranasal delivery system according to claim 1, and wherein the intranasal delivery system comprises immunoglobulins effective against multitude of strains of the virus.
